(19) United States
(12) Patent Application Publication
(10) Pub. No.: US 2008/0242700 A1
(43) Pub. Date: Oct. 2, 2008

(54) Methods and Compositions for Selectin Inhibition

(75) Inventors: Neelu Kaila, Lexington, MA (US); Adrian Huang, Lexington, MA (US); Patricia Ward Bedard, Foxboro, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/058,396

(22) Filed: Mar. 28, 2008

Related U.S. Application Data
(60) Provisional application No. 60/920,950, filed on Mar. 30, 2007.

(51) Int. Cl. A61K 31/47; C07D 215/20; A61P 9/00

(52) U.S. Cl. 514/312; 546/154

(57) ABSTRACT

The present teachings relate to compounds of formula I:

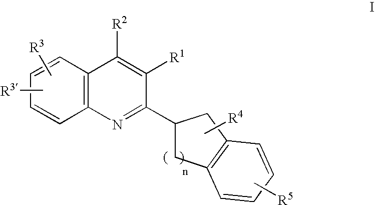

wherein the constituent variables are defined herein. Compounds of the present teachings can act as antagonists of the mammalian adhesion proteins known as selecting. Methods for treating selectin mediated disorders are provided, which include administration of these compounds in a therapeutically effective amount.

This application claims the benefit of U.S. Provisional Application No. 60/920950, filed Mar. 30, 2007, the entire disclosure of which is incorporated by reference herein.

### BACKGROUND

The present teachings relate to novel compounds that act as antagonists of the mammalian adhesion proteins known as selecting.

During the initial phase of vascular inflammation, leukocytes and platelets in flowing blood decrease velocity by adhering to the vascular endothelium and by exhibiting rolling behavior. This molecular tethering event is mediated by specific binding of a family of calcium-dependent or “C-type” lectins, known as selectins, to ligands on the surface of leukocytes. There are also several disease states that can cause the deleterious triggering of selectin-mediated cellular adhesion, such as autoimmunity disorders, thrombotic disorders, parasitic diseases, and metastatic spread of tumor cells.

The extracellular domain of a selectin protein is characterized by an N-terminal lectin-like domain, an epidermal growth factor-like domain, and varying numbers of short consensus repeats. Three human selectin proteins have been identified, including P-selectin (formerly known as PADGEM or GMP-140), E-selectin (formerly known as ELAM-1), and L-selectin (formerly known as LAM-1). E-selectin expression is induced on endothelial cells by proinflammatory cytokines via its transcriptional activation. L-selectin is constitutively expressed on leukocytes and appears to play a key role in lymphocyte homing. P-selectin is stored in the alpha granules of platelets and the Weibel-Palade bodies of endothelial cells and therefore can be rapidly expressed on the surface of these cell types in response to proinflammatory stimuli. Selectins mediate adhesion through specific interactions with ligand molecules on the surface of leukocytes. Generally, the ligands of selectins are comprised, at least in part, of a carbohydrate moiety. For example, E-selectin binds to carbohydrates having the terminal structure:

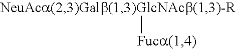

and also to carbohydrates having the terminal structures:

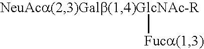

wherein R is the remainder of the carbohydrate chain. These carbohydrates are known blood group antigens and are commonly referred to as Sialyl Lewis x and Sialyl Lewis a, respectively. The presence of the Sialyl Lewis x antigen alone on the surface of an endothelial cell may be sufficient to promote binding to an E-selectin expressing cell. E-selectin also binds to carbohydrates having the terminal structures:

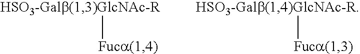

As with E-selectin, each selectin appears to bind to a range of carbohydrates with varying affinities. The strength of the selectin-mediated adhesive event (binding affinity) may also depend on the density and context of the selectin on the cell surface.

Structurally diverse glycoprotein ligands, including GlyCAM-1, CD34, ESL-1, and PSGL-1 can bind to selectins with apparent high affinity. PSGL-1 is a mucin-like homodimeric glycoprotein expressed by virtually all subsets of leukocytes and is recognized by each of the three selectins. However, PSGL-1 appears to be unique in that it is the predominant high affinity P-selectin ligand on leukocytes. High affinity P-selectin binding to PSGL-1 requires both an sLex-containing O-glycan and one or more tyrosine sulfate residues within the anionic N-terminus of the PSGL-1 polypeptide (see Somers, W. S. et al., Cell, 2000, 103: 467-479; Sako, D. et al., Cell, 1995, 82(2): 323-331; Pouyani, N. et al., Cell, 1995, 82(2): 333-343; and Wilkins, P. P. et al., J. Biol. Chem., 1995, 270(39): 22677-22680). L-Selectin also recognizes the N-terminal region of PSGL-1 and has similar sulfation-dependent binding requirements to that of P-selectin. The ligand requirements of E-selectin appear to be less stringent as it can bind to the sLex-containing glycans of PSGL-1 and other glycoproteins. Despite the fact that P-selectin knockout and P/E selectin double knockout mice show elevated levels neutrophils in the blood, these mice show an impaired DTH response and delayed thioglycolate-induced peritonitis (TIP) response (see Frenette, P. S. et al., Thromb Haemost, 1997, 78(1): 60-64). Soluble forms of PSGL-1 such as rPSGL-Ig have shown efficacy in numerous animal models (see Kumar, A. et. al., Circulation, 1999, 99(10): 1363-1369; Takada, M. et. al., J. Clin. Invest., 1997, 99(11): 2682-2690; and Scalia, R. et al., Circ Res., 1999, 84(1): 93-102).

In addition, P-selectin ligand proteins, and the genes encoding the same, have been identified. See U.S. Pat. No. 5,840,679. As demonstrated by P-selectin/LDLR deficient mice, inhibition of P-selectin represents a useful target for the treatment of atherosclerosis (see Johnson, R. C. et al., J. Clin. Invest, 1997, 99: 1037-1043). An increase in P-selectin expression has been reported at the site of atherosclerotic lesions, and the magnitude of the P-selectin expression appears to correlate with the lesion size. It is likely that the adhesion of monocytes, mediated by P-selectin, contributes to atherosclerotic plaque progression (see Molenaar, T. J. M. et al., Biochem. Pharmacol., 2003, (66): 859-866).

Inhibition of P-selectin may also represent a useful target for other diseases or conditions, including, for example, thrombosis (Wakefield et al., Arterioscler Thromb Vasc Biol 28 (2008) 387-391; Myers et al., Thromb Haemost 97 (2007) 400-407), atherothrombosis (Fuster et al., Journal of the American College of Cardiology 46 (2005) 1209-1218), restenosis (Bienvenu et al., Circulation 103 (2001) 1128-1134), myocardial infarction (Furman et al., Journal of the American College of Cardiology 38 (2001) 1002-1006), ischemia reperfusion, Reynauld's syndrome, inflammatory bowel disease, osteoarthritis, acute respiratory distress syndrome, asthma (Romano, Treat Respir Med 4 (2005) 85-94), chronic obstructive pulmonary disease (Romano, Treat Respir Med 4 (2005) 85-94), emphysema, lung inflammation, delayed type hyper-sensitivity reaction (Staite et al., Blood 88 (1996) 2973-2979), idiopathic pulmonary fibrosis, cystic fibrosis, thermal injury, stroke, experimental allergic encephalomyelitis, multiple organ injury syndrome secondary to trauma, neutrophilic dermatosis (Sweet's disease), glomerulonephritis (Tianfu Wu, Arthritis & Rheumatism 56 (2007) 949-959), ulcerative colitis (Irving et al., European Journal of Gastroenterology & Hepatology 20 (2008) 283-289), Crohn's disease, necrotizing enterocolitis, cytokine-induced toxicity, gingivitis (Krugluger et al., J Periodontal Res 28: 145-151), periodontitis (Krugluger et al., J Periodontal Res 28: 145-151), hemolytic uremic syndrome, psoriasis (Friedrich et al., Archives of Dermatological Research 297 (2006) 345-351), systemic lupus erythematosus, autoimmune thyroiditis, multiple sclerosis, rheumatoid arthritis (Grober et al., J. Clin. Invest. 91 (1993) 2609-2619), Grave's disease (Hara et al., Endocr J. 43 (1996) 709-713), immunological-mediated side effects of treatment associated with hemodialysis or leukapheresis, granulocyte transfusion associated syndrome, deep vein thrombosis (Myers et al., Thromb Haemost 97 (2007) 400-407), post-thrombotic syndrome, unstable angina, transient ischemic attacks, peripheral vascular disease (e.g., peripheral arterial disease) (van der Zee et al., Clin Chem 52 (2006) 657-664), metastasis associated with cancer (McEver, Glycoconjugate Journal 14 (1997) 585-591), sickle syndromes (including but not limited to sickle cell anemia) (Blann et al., Journal of Thrombosis and Thrombolysis, 10.1007/s11239-007-0177-7 (Dec. 14, 2007)), organ rejection (graft vs. host), or congestive heart failure.

Given the role of selectins in numerous important biological processes, including inflammation and adhesion processes, it can be seen that there is a continuing need for new selectin inhibitors.

### SUMMARY

The present teachings provide compounds of formula I:

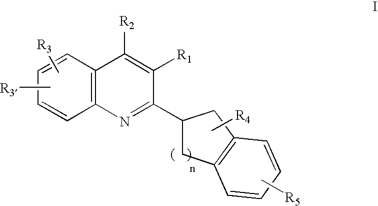

and pharmaceutically acceptable salts, hydrates, and esters thereof, wherein R₁, R₂, R₃, R_{3′}, R₄, R₅, and n are as defined herein.

The present teachings also relate to pharmaceutical compositions that include a pharmaceutically effective amount of one or more compounds of formula I (or their pharmaceutically acceptable salts, hydrates, or esters) and a pharmaceutically acceptable carrier or excipient. The present teachings also provide methods of making and using the compounds of formula I and their pharmaceutically acceptable salts, hydrates, and esters. In some embodiments, the present teachings provide methods of treating mammals having conditions characterized by selectin-mediated intercellular adhesion processes, for example, by administering to the mammal an effective amount of one or more compounds of formula I or their pharmaceutically acceptable salts, hydrates, and esters, to at least partially modulate selectin-mediated intracellular adhesion in a mammal.

### DETAILED DESCRIPTION

The present teachings provide compounds of formula I:

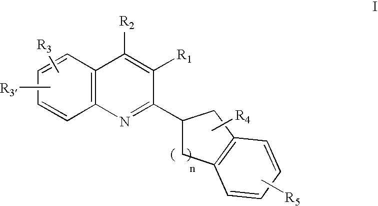

and pharmaceutically acceptable salts, hydrates, and esters thereof, wherein:
R₁ is —OR₆, —C(O)R₇, —C(O)OR₆, —C(O)NR₇R₈, —C(S)R₇, —C(S)OR₆, —C(S)NR₇R₈, —C(NR₇)R₇, —C(NR₇)NR₇R₈, —NR₇R₈, —NR₈C(O)R₇, —NR₈C(O)NR₇R₈, —NR₈C(NR₇)NR₇R₈, —NR₈S(O)ₘR₇, or —NR₈S(O)ₘNR₇R₈;
R₂ is —C(O)OR₆, —C(O)NR₇R₈, or a carboxylic acid bioisostere;
R₃ and R₃ independently are H, —CN, —NO₂, halogen, —OR₆, —NR₇R₈, —S(O)ₘR₇, —S(O)ₘOR₆, —S(O)ₘNR₇R₈, —C(O)R₇, —C(O)OR₆, —C(O)NR₇R₈, —C(S)R₇, —C(S)OR₆, —C(S)NR₇R₈, —C(NR₇)NR₇R₈, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₉ groups; or
alternatively, R₃ and R₃, together with the carbon atoms to which each is attached, form a C₄₋₁₄ cycloalkyl group, a C₆₋₁₄ aryl group, a 4-14 membered cycloheteroalkyl group, or a 5-14 membered heteroaryl group, wherein each of the C₄₋₁₄ cycloalkyl group, the C₆₋₁₄ aryl group, the 4-14 membered cycloheteroalkyl group, and the 5-14 membered heteroaryl group optionally is substituted with 1-4-Z-R₉ groups;
R₄ and R₅ independently are H, —CN, —NO₂, halogen, —OR₆, —NR₇R₈, —S(O)ₘR₇, —S(O)ₘOR₆, —S(O)ₘNR₇R₈, —C(O)R₇, —C(O)OR₆, —C(O)NR₇R₈, —C(S)R₇, —C(S)OR₆, —C(S)NR₇R₈, —C(NR₇)NR₇R₈, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the —C(NR₇)NR₇R₈, the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₉ groups;
R₆, at each occurrence, independently is H, —C(O)R₇, —C(O)NR₇R₈, —C(S)R₇, —C(S)NR₇R₈, —C(NR₇)R₇, —C(NR₇)NR₇R₈, —S(O)ₘR₇, —S(O)ₘNR₇R₈, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, or the 5-14 membered heteroaryl group optionally is substituted with 1-4-Z-R₉ groups;
R₇ and R₈, at each occurrence, independently are H, —OH, —SH, —S(O)₂OH, —C(O)OH, —C(O)NH₂, —C(S)NH₂, —OC₁₋₁₀ alkyl, —C(O)—C₁₋₁₀ alkyl, —C(O)—OC₁₋₁₀ alkyl, —OC₆₋₁₄ aryl, —C(O)—C₆₋₁₄ aryl, —C(O)—OC₆₋₁₄ aryl, —C(S)N(C₁₋₁₀ alkyl)₂, —C(S)NH—C₁₋₁₀ alkyl, —C(O)NH—C₁₋₁₀ alkyl, —C(O)N(C₁₋₁₀ alkyl)₂, —C(O)NH—C₆₋₁₄ aryl, —S(O)ₘ—C₁₋₁₀ alkyl, —S(O)ₘ—OC₁₋₁₀ alkyl, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₉ groups;
R₉, at each occurrence, independently is halogen, —CN, —NO₂, oxo, —O-Z-R₁₀, —NR₁₀-Z-R₁₁, —N(O)R₁₀-Z-R₁₁, —S(O)ₘR₁₀, —S(O)ₘO-Z-R₁₀, —S(O)ₘNR₁₀-Z-R₁₁, —C(O)R₁₀, —C(O)O-Z-R₁₀, —C(O)NR₁₀-Z-R₁₁, —C(S)NR₁₀-Z-R₁₁, —Si(Cl₁₋₁₀ alkyl)₃, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₁₂ groups;
R₁₀ and R₁₁, at each occurrence, independently are H, —OH, —SH, —S(O)₂OH, —C(O)OH, —C(O)NH₂, —C(S)NH₂, —OC₁₋₁₀ alkyl, —C(O)—C₁₋₁₀ alkyl, —C(O)—OC₁₋₁₀ alkyl, —C(S)N(C₁₋₁₀ alkyl)₂, —C(S)NH—C₁₋₁₀ alkyl, —C(O)NH—C₁₋₁₀ alkyl, —C(O)N(C₁₋₁₀ alkyl)₂, —S(O)ₘ-C₁₋₁₀ alkyl, —S(O)ₘ—OC₁₋₁₀ alkyl, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₁₂ groups;
R₁₂, at each occurrence, independently is halogen, —CN, —NO₂, oxo, —OH, —NH₂, —NH(C₁₋₁₀ alkyl), —N(C₁₋₁₀ alkyl)₂, —S(O)ₘH, j) —S(O)ₘ-C₁₋₁₀ alkyl, —S(O)₂OH, —S(O)ₘ—OC₁₋₁₀ alkyl, —CHO, —C(O)—C₁₋₁₀ alkyl, —C(O)OH, —C(O)—OC₁₋₁₀ alkyl, —C(O)NH₂, —C(O)NH—C₁₋₁₀ alkyl, —C(O)N(C₁₋₁₀ alkyl)₂, —C(S)NH₂, —C(S)NH—C₁₋₁₀ alkyl, —C(S)N(C₁₋₁₀ alkyl)₂, —S(O)ₘNH₂, —S(O)ₘNH(C₁₋₁₀ alkyl), —S(O)ₘN(C₁₋₁₀ alkyl)₂, —Si(C₁₋₁₀ alkyl)₃, a C₁₋₁₀ alkyl group, a C₂₋₁₀ alkenyl group, a C₂₋₁₀ alkynyl group, a C₁₋₁₀ alkoxy group, a C₁₋₁₀ alkylthio group, a C₁₋₁₀ haloalkyl group, a C₃₋₁₄ cycloalkyl group, a C₆₋₁₄ aryl group, a 3-14 membered cycloheteroalkyl group, or a 5-14 membered heteroaryl group;
Z, at each occurrence, independently is a divalent C₁₋₁₀ alkyl group, a divalent C₂₋₁₀ alkenyl group, a divalent C₂₋₁₀ alkynyl group, a divalent C₁₋₁₀ haloalkyl group, or a covalent bond;
m, at each occurrence, independently is 0, 1, or 2; and
n is 0, 1, or 2.

In some embodiments, R₁ can be —OR₆ or —NR₇R₈, wherein R₆ can be H, —C(O)R₇, —C(O)NR₇R₈, —C(S)R₇, —C(S)NR₇R₈, —S(O)ₘR₇, —S(O)ₘNR₇R₈, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group can be optionally substituted with 1-4-Z-R₉ groups, and R₇, R₈, R₉, Z, and m are as defined herein. For example, R₁ can be —OH, —OC(O)R₇, —OC(O)NR₇R₈, —OS(O)ₘR₇, —OS(O)ₘNR₇R₈, or —NR₇R₈. In certain embodiments, R₁ can be —OH, —OC(O)R₇, or —NR₇R₈. In particular embodiments, R₁ can be —OH.

In some embodiments, R₂ can be —C(O)OR₆, wherein R₆ is as defined herein. In certain embodiments, R₆ can be H, a C₁₋₁₀ alkyl group, a C₂₋₁₀ alkenyl group, a C₂₋₁₀ alkynyl group, a C3-14 cycloalkyl group, a C₆₋₁₄ aryl group, a 3-14 membered cycloheteroalkyl group, or a 5-14 heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group can be independently and optionally substituted with 1-4 -Z-R₉ groups, and Z and R₉ are as defined herein. For example, R₂ can be —C(O)OH.

In other embodiments, R₂ can be —C(O)NR₁₀R₁₁, wherein R₁₀ and R₁₁ are as defined herein. For example, R₁₀ and R₁₁ independently can be H, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₁₂ groups. In particular embodiments, R₂ can be —C(O)NH₂ or —C(O)NHR₁₀, wherein R₁₀ can be a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₃₋₁₄ cycloalkyl group, the C₆₋₁₄ a group, the 3-14 membered cycloheteroalkyl group, and the 5-14 membered heteroaryl group optionally is substituted with 1-4-Z-R₁₂ groups.

In other embodiments, R₂ can be a carboxylic acid bioisostere, such as, but not limited to, an amide, a sulfonamide, a sulfonic acid, 3-hydroxy-4H-pyran-4-one, an imidazole, an oxazole, a thiazole, a pyrazole, a triazole, an oxadiazole, a thiadiazole, or a tetrazole, each of which optionally can be substituted (e.g., by a C₁₋₁₀ alkyl group, OH, etc.).

In some embodiments, compounds of the present teachings can be represented by formula Ia, formula Ib, formula Ic, formula Id, formula Ie, or formula If:

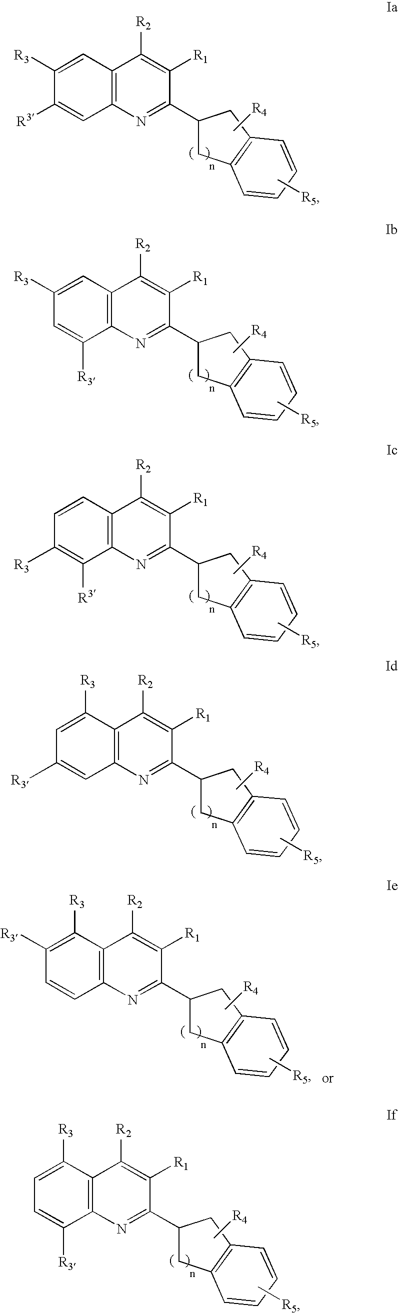

wherein R₁, R₂, R₃, R₃, R₄, R₅, and n are as defined herein.

For compounds of formula I, formula Ia, formula Ib, formula Ic, formula Id, formula Ie, or formula If, R₃ and R_{3′}, in some embodiments, independently can be H, halogen, —OR₆, —C(O)OR₆, a C₁₋₁₀ alkyl group, a C₃₋₁₄ cycloalkyl group, a C₆₋₁₄ aryl group, or a 5-14 membered heteroaryl group, wherein each of the C₁₋₁₀ alkyl group, the C₃₋₁₄ cycloalkyl group, the C₆₋₁₄ aryl group, and the 5-14 membered heteroaryl group can be optionally substituted with 1-4-Z-R₉ groups, and Z and R₉ are as defined herein. In certain embodiments, R₃ and R_{3′} independently can be H, F, Cl, Br, —OH, —O(C₁₋₆ alkyl), —COOH, a C₁₋₆ alkyl group, a C₃₋₁₀ cycloalkyl, a phenyl group, or a 5-10 membered heteroaryl group, wherein each of the C₁₋₆ alkyl group, the C₃₋₁₀ cycloalkyl group, the phenyl group, and the 5-10 membered heteroaryl group can be optionally substituted with 1-4-Z-R₉ groups, and Z and R₉ are as defined herein. For example, R₃ and R_{3′} can independently be —O—(C₁₋₆ alkyl), wherein the C₁₋₆ alkyl group can be optionally substituted (e.g., —OCH₃, —OCH₂CH₃, —OCH(CH₃)₂, —OCH₂CH₂CH₃, —OC(CH₃)₃, and —OCF₃), an optionally substituted straight-chain or branched C₁₋₆ alkyl group (e.g. a methyl group, an ethyl group, a n-propyl group, an iso-propyl group, a n-butyl group, a sec-butyl group, a tert-butyl group, —CF₃, —C(CH₃)₂OH, —C(CF₃)(CH₃)OH, and —C(CF₃)₂OH), or an optionally substituted C₃₋₁₄ cycloalkyl group (e.g., a cyclopropyl group, a cyclobutyl group, a cyclopentyl group, a cyclohexyl group, and a cycloheptyl group). In some embodiments, R₃ and R_{3′} can independently be H, —C(CH₃)₂OH, —C(CF₃)(CH₃)OH, or —C(CF₃)₂OH. In some embodiments, R₃ can be H and R_{3′} can be —C(CF₃)₂OH. In other embodiment, R₃ can be —C(CF₃)₂OH and R₃ can be H. In other embodiments, R₃ and R_{3′} can both be H. In certain embodiments, R₃ or R_{3′} can be a phenyl group or a thienyl group, each of which can be optionally substituted with 1-4-Z-R₉ groups, and Z and R₉ are as defined herein.

In other embodiments, R₃ and R_{3′}, together with the carbon atoms to which each is attached, can form a C₄₋₁₄ cycloalkyl group or a 4-14 membered cycloheteroalkyl group, wherein each of the C₄₋₁₄ cycloalkyl group and the 4-14 membered cycloheteroalkyl group can be optionally substituted with 1-4-Z-R₉ groups, and Z and R₉ are as defined herein. Examples of cycloalkyl groups and cycloheteroalkyl groups include, but are not limited to, a cyclohexyl group and a piperidyl group, each of which can be optionally substituted with 1-4-Z-R₉ groups, and Z and R₉ are as defined herein. For example, R₃ and R_{3′}, together with the carbon atoms to which they are attached, can form a cyclohexyl group. In some embodiments, compounds of the present teachings have formula Ig:

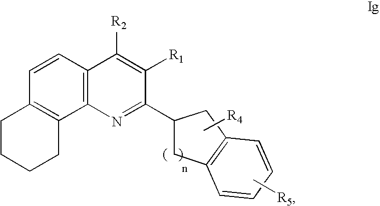

wherein R¹, R², R⁴, R⁵ and n are as defined herein.

In some embodiments of the compounds of the present teachings, n can be 0. In other embodiments, n can be 1.

In some embodiments, R₄ can be H, —CN, —NO₂, halogen, —OR₆, —NR₇R₈, —S(O)ₘR₇, —S(O)ₘOR₆, —S(O)ₘNR₇R₈, —C(O)R₇, —C(O)OR₆, —C(O)NR₇R₈, or a C₁₋₁₀ alkyl group optionally substituted with 1-4-Z-R₉ groups; wherein R₆, R₇, R₈, R₉, and Z are as defined herein. In some embodiments, R₄ can be H, —CN, —NO₂, halogen, —OH, —NH₂, —C(O)OH, —C(O)NH₂, —O(C₁₋₁₀ alkyl), —NH(C₁₋₁₀ alkyl), —N(C₁₋₁₀ alkyl)₂, —C(O)O(C₁₋₁₀ alkyl), —C(O)NH(C₁₋₁₀ alkyl), —C(O)N(C₁₋₁₀ alkyl)₂, or a C₁₋₁₀ alkyl group optionally substituted with 1-4-Z-R₉ groups; wherein R₉ and Z are as defined herein. In particular embodiments, R₄ can be H.

In some embodiments, R₅ can be H, —CN, —NO₂, halogen, —OR₆, —NR₇R₈, —S(O)ₘR₇, —S(O)ₘOR₆, —S(O)ₘNR₇R₈, —C(O)R₇, —C(O)OR₆, —C(O)NR₇R₈, or a C₁₋₁₀ alkyl group optionally substituted with 1-4-Z-R₉ groups; wherein R₆, R₇, R₈, R₉, and Z are as defined herein. In some embodiments, R₅ can be H, —CN, —NO₂, halogen, —OH, —NH₂, —C(O)OH, —C(O)NH₂, —O(C₁₋₁₀ alkyl), —NH(C₁₋₁₀ alkyl), —N(C₁₋₁₀ alkyl)₂, —C(O)O(C₁₋₁₀ alkyl), —C(O)NH(C₁₋₁₀ alkyl), —C(O)N(C₁₋₁₀ alkyl)₂, or a C₁₋₁₀ alkyl group optionally substituted with 1-4-Z-R₉ groups; wherein R₉ and Z are as defined herein. In particular embodiments, R₅ can be H.

In some embodiments, compounds of the present teachings can be represented by formula IIa or IIb:

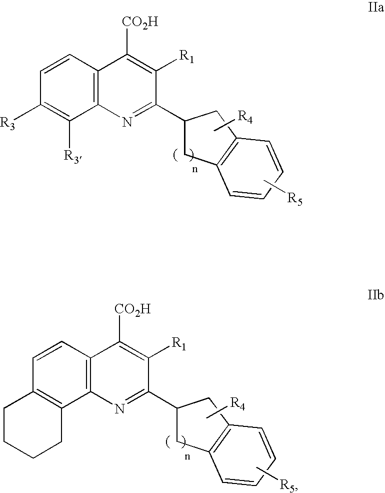

wherein R₁, R₃, R_{3′}, R₄, R₅, and n are as defined herein above.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, “halo” or “halogen” refers to fluoro, chloro, bromo, and iodo.

As used herein, “oxo” refers to a double-bonded oxygen (i.e., ═O).

As used herein, “alkyl” refers to a straight-chain or branched saturated hydrocarbon group. Examples of alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) groups, and the like. In some embodiments, alkyl groups can be substituted with up to four substituents independently selected from -Z-R₉ and -Z-R₁₂ groups, wherein Z, R₉, and R₁₂ are as described herein. A lower alkyl group typically has up to 6 carbon atoms. Examples of lower alkyl groups include methyl, ethyl, propyl (e.g., n-propyl and isopropyl), and butyl groups (e.g., n-butyl, isobutyl, s-butyl, t-butyl).

As used herein, “alkenyl” refers to a straight-chain or branched alkyl group having one or more carbon-carbon double bonds. Examples of alkenyl groups include, but are not limited to, ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In some embodiments, alkenyl groups can be substituted with up to four substituents independently selected from -Z-R₉ and -Z-R₁₂ groups, wherein Z, R₉, and R₁₂ are as described herein.

As used herein, “alkynyl” refers to a straight-chain or branched alkyl group having one or more carbon-carbon triple bonds. Examples of alkynyl groups include, but are not limited to, ethynyl, propynyl, butynyl, pentynyl, and the like. The one or more carbon-carbon triple bonds can be internal (such as in 2-butyne) or terminal (such as in 1-butyne). In some embodiments, alkynyl groups can be substituted with up to four substituents independently selected from -Z-R₉ and -Z-R₁₂ groups, wherein Z, R₉, and R₁₂ are as described herein.

As used herein, “alkoxy” refers to an —O-alkyl group. Examples of alkoxy groups include, but are not limited to, methoxy, ethoxy, propoxy (e.g., n-propoxy and isopropoxy), t-butoxy groups, and the like. In some embodiments, the alkyl group in an —O-alkyl group can be substituted with up to four substituents independently selected from -Z-R₉ and -Z-R₁₂ groups, wherein Z, R₉, and R₁₂ are as described herein.

As used herein, “alkylthio” refers to an —S-alkyl group. Examples of alkylthio groups include, but are not limited to, methylthio, ethylthio, propylthio (e.g., n-propylthio and isopropylthio), t-butylthio groups, and the like. In some embodiments, the alkyl group in an —S-alkyl group can be substituted with up to four substituents independently selected from -Z-R₉ and -Z-R₁₂ groups, wherein Z, R₉, and R₁₂ are as described herein.

As used herein, “haloalkyl” refers to an alkyl group having one or more halogen substituents. Examples of haloalkyl groups include, but are not limited to, CF₃, C₂F₅, CHF₂, CH₂F, CCl₃, CHCl₂, CH₂Cl, C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.”

As used herein, “cycloalkyl” refers to a non-aromatic carbocyclic group including cyclized alkyl, alkenyl, and alkynyl groups, e.g., having from 3 to 14 ring carbon atoms and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Examples of cycloalkyl groups include, but are not limited to, cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like. In some embodiments, cycloalkyl groups can be substituted with up to four substituents independently selected from -Z-R₉ and -Z-R₁₂ groups, wherein Z, R₉, and R₁₂ are as described herein. In some embodiments, cycloalkyl groups can be substituted with one or more oxo groups.

As used herein, “heteroatom” refers to an atom of any element other than carbon or hydrogen and includes, for example, nitrogen (N), oxygen (O), sulfur (S), phosphorus (P), and selenium (Se).

As used herein, “cycloheteroalkyl” refers to a non-aromatic cycloalkyl group having 3-14 ring atoms that contains at least one ring heteroatom (e.g., 1-5) selected from O, N, and S, and optionally contains one or more (e.g., 1, 2, or 3) double or triple bonds. The cycloheteroalkyl group can be attached to the defined chemical structure at any heteroatom or carbon atom that results in a stable structure. One or more N or S atoms in a cycloheteroalkyl ring can be oxidized (e.g., morpholine N-oxide, thiomorpholine S-oxide, thiomorpholine S,S-dioxide). In some embodiments, nitrogen atoms of cycloheteroalkyl groups can bear a substituent, for example, a -Z-R₉ or -Z-R₁₂ groups, wherein Z, R₉, and R₁₂ are as described herein. Cycloheteroalkyl groups can also contain one or more oxo groups, such as phthalimide, piperidone, oxazolidinone, pyrimidine-2,4(1H,3H)-dione, pyridin-2(1H)-one, and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothienyl, piperidinyl, piperazinyl groups, and the like. In some embodiments, cycloheteroalkyl groups can be optionally substituted with up to four substituents independently selected from -Z-R₉ and -Z-R₁₂ groups, wherein Z, R₉, and R₁₂ are as described herein.

As used herein, “aryl” refers to an aromatic monocyclic hydrocarbon ring system or a polycyclic ring system having an aromatic monocyclic hydrocarbon ring fused to at least one other aromatic hydrocarbon ring and/or non-aromatic carbocyclic or heterocyclic ring. In some embodiments, a monocyclic aryl group can have from 6 to 14 carbon atoms and a polycyclic aryl group can have from 8 to 14 carbon atoms. Any suitable ring position of the aryl group can be covalently linked to the defined chemical structure. In some embodiments, an aryl group can have only aromatic carbocyclic rings, e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cycloalkyl or cycloheteroalkyl rings. Examples of such aryl groups include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include, but are not limited to, benzodioxanyl, benzodioxolyl, chromanyl, indolinyl groups, and the like. In some embodiments, aryl groups can optionally contain up to four substituents independently selected from -Z-R₉ and -Z-R₁₂ groups, wherein Z, R₉, and R₁₂ are as described herein.

As used herein, “heteroaryl” refers to an aromatic monocyclic ring system containing at least 1 ring heteroatom selected from oxygen (O), nitrogen (N), and sulfur (S) or a polycyclic ring system where at least one of the rings present in the ring system is aromatic and contains at least 1 ring heteroatom. A heteroaryl group, as a whole, can have, for example, from 5 to 14 ring atoms and contain 1-5 ring heteroatoms. Heteroaryl groups include monocyclic heteroaryl rings fused to one or more aromatic carbocyclic rings, non-aromatic carbocyclic rings, and non-aromatic cycloheteroalkyl rings. The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5-membered monocyclic and 5-6 bicyclic ring systems shown below:

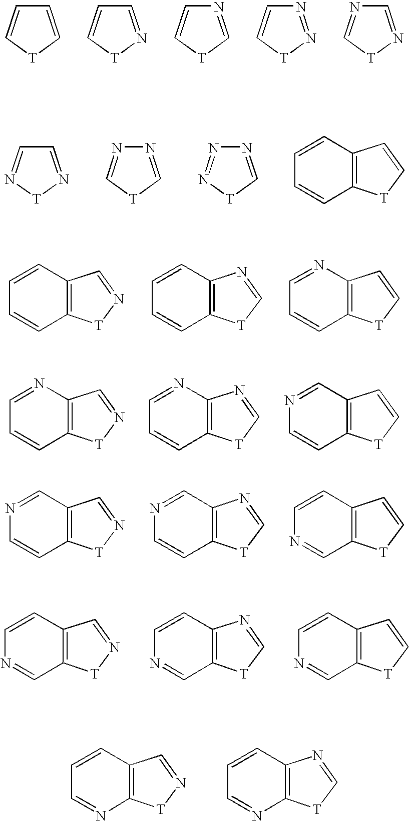

wherein T is O, S, NH, N-Z-R₉ , or N-Z-R₁₂, wherein Z, R₉, and R₁₂ are defined as herein. Examples of such heteroaryl rings include, but are not limited to, pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include, but are not limited to, 4,5,6,7-tetrahydroindolyl, tetrahydroquinolinyl, benzothienopyridinyl, benzofuropyridinyl groups, and the like. In some embodiments, heteroaryl groups can be substituted with up to four substituents independently selected from -Z-R₉ and -Z-R₁₂ groups, wherein Z, R₉, and R₁₂ are as described herein.

As used herein, “carboxylic acid bioisostere” refers to a substituent or group that has chemical or physical properties similar to that of a carboxylic acid moiety and that produces broadly similar biological properties to that of a carboxylic acid moiety. See generally, R. B. Silverman, The Organic Chemistry of Drug Design and Drug Action (Academic Press, 1992). Examples of carboxylic acid bioisosteres include, but are not limited to, amides, sulfonamides, sulfonic acids, phosphonamidic acids, alkyl phosphonates, N-cyanoacetamides, 3-hydroxy-4H-pyran-4-one, imidazoles, oxazoles, thiazoles, pyrazoles, triazoles, oxadiazoles, thiadiazoles, or tetrazoles, each of which optionally can be substituted (e.g., by a C₁₋₁₀ alkyl group, OH, etc.). Other examples of carboxylic acid bioisostere can include, but are not limited to, —OH and those shown below:

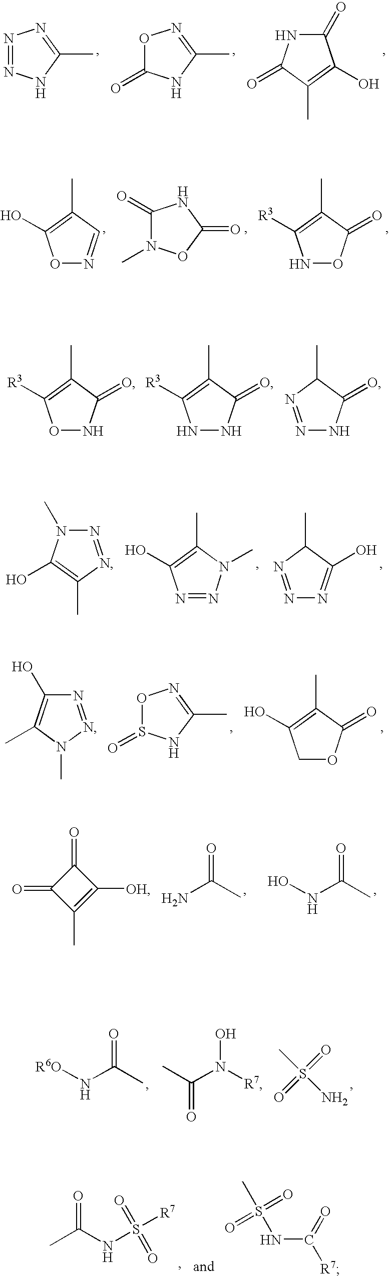

wherein R₃, R₆, and R₇ are defined as herein.

Compounds of the present teachings can include a “divalent group” defined herein as a linking group capable of forming a covalent bond with two other moieties. For example, compounds described herein can include a divalent C₁₋₁₀ alkyl group, such as, for example, a methylene group.

At various places in the present specification, substituents of compounds are disclosed in groups or in ranges. It is specifically intended that the description include each and every individual subcombination of the members of such groups and ranges. For example, the term “C₁₋₁₀ alkyl” is specifically intended to individually disclose C₁, C₂, C₃, C₄, C₅, C₆, C₇, C₈, C₉, C₁₀, C₁-C₁₀, C₁-C₉, C₁-C₈, C₁-C₇, C₁-C₆, C₁-C₅, C₁-C₄, C₁-C₃, C₁-C₂, C₂-C₁₀, C₂-C₉, C₂-C₈, C₂-C₇, C₂-C₆, C₂-C₅, C₂-C₄, C₂-C₃, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉, and C₉-C₁₀ alkyl. By way of another example, the term “5-14 membered heteroaryl group” is specifically intended to individually disclose a heteroaryl group having 5, 6, 7, 8, 9, 10, 11, 12, 13, 14, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, or 13-14 ring atoms.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such optical isomers (enantiomers) and diastereomers (geometric isomers), as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Throughout the specification, structures may or may not be presented with chemical names. Where any question arises as to nomenclature, the structure prevails.

Also provided in accordance with the present teachings are prodrugs of compounds disclosed herein. As used herein, “prodrug” refers to a moiety that produces, generates or releases a compound of the present teachings when administered to a mammalian subject. Prodrugs can be prepared by modifying functional groups present in the compounds in such a way that the modifications are cleaved, either by routine manipulation or in vivo, from the parent compounds. Examples of prodrugs include compounds as described herein that contain one or more molecular moieties appended to a hydroxyl, amino, sulfhydryl, or carboxyl group of the compound, and that when administered to a mammalian subject, is cleaved in vivo to form the free hydroxyl, amino, sulfhydryl, or carboxyl group, respectively. Examples of prodrugs can include, but are not limited to, acetate, formate, and benzoate derivatives of alcohol and amine functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosures of which are incorporated by reference herein for all purposes.

Ester forms of the compounds according to the present teachings include pharmaceutically acceptable esters known in the art, which can be metabolized into the free acid form, such as a free carboxylic acid form, in a mammal body. Examples of suitable esters include, but are not limited to alkyl esters (e.g., of 1 to 10 carbon atoms), cycloalkyl esters (e.g., of 3-10 carbon atoms), aryl esters (e.g., of 6-14 carbon atoms, including of 6-10 carbon atoms), and heterocyclic analogues thereof (e.g., of 3-14 ring atoms, 1-3 of which can be selected from oxygen, nitrogen, and sulfur heteroatoms) and the alcoholic residue can carry further substituents. In some embodiments, esters of the compounds disclosed herein can be C₁₋₁₀ alkyl esters, such as methyl esters, ethyl esters, propyl esters, isopropyl esters, butyl esters, isobutyl esters, t-butyl esters, pentyl esters, isopentyl esters, neopentyl esters, and hexyl esters, C₃₋₁₀ cycloalkyl esters, such as cyclopropyl esters, cyclopropylmethyl esters, cyclobutyl esters, cyclopentyl esters, and cyclohexyl esters, or aryl esters, such as phenyl esters, benzyl esters, and tolyl esters.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di-, or tri-lower alkylamine (e.g., ethyl-tert-butyl-, diethyl-, diisopropyl-, triethyl-, tributyl-, or dimethylpropylamine), or a mono-, di-, or trihydroxy lower alkylamine (e.g., mono-, di-, or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, naphthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known anti-inflammatory agents. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal, or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes, or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal, and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age, and response pattern of the patient.

In some cases, for example those in which the lung is the targeted organ, it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can be sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal). Topical formulations that deliver compound(s) of the present teachings through the epidermis can be useful for localized treatment of inflammation, psoriasis, and arthritis.

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human. The present teachings accordingly provide methods of treating or inhibiting a pathological condition or disorder by providing to a mammal a compound of the present teachings (or its pharmaceutically acceptable salt, hydrate, or ester) or a pharmaceutical composition that includes a compound of the present teachings in combination or association with one or more pharmaceutically acceptable carriers. Compounds of the present teachings can be administered alone or in combination with other therapeutically effective compounds or therapies for the treatment or inhibition of the pathological condition or disorder. As used herein, “therapeutically effective” refers to a substance or an amount that elicits a desirable biological activity or effect. As used herein, “treating” refers to partially or completely alleviating, inhibiting, and/or ameliorating the condition.

The present teachings further include use of the compounds disclosed herein and their pharmaceutically acceptable salts, hydrates, and esters as active therapeutic substances for the treatment or inhibition of a pathological condition or disorder in a mammal. In some embodiments, the pathological condition or disorder can be associated with selectin-mediated intracellular adhesion. Accordingly, the present teachings further provide methods of treating these pathological conditions and disorders using the compounds described herein.

In some embodiments, the present teachings provide methods of inhibiting selectin-mediated intracellular adhesion in a mammal that include administering to the mammal an effective amount of a compound of the present teachings or its pharmaceutically acceptable salt, hydrate, or ester. In certain embodiments, the present teachings provide methods of inhibiting selectin-mediated intracellular adhesion associated with a disease, disorder, condition, or undesired process in a mammal, that include administering to the mammal a therapeutically effective amount of a compound disclosed herein.

In some embodiments, the disease, disorder, condition, or undesired process can be infection, metastasis, an undesired immunological process, an undesired thrombotic process, or a disease or condition with an inflammatory component (e.g., cardiovascular disease, diabetes, or rheumatoid arthritis). In some embodiments, the disease, disorder, condition, or undesired process can be atherosclerosis, atherothrombosis, restenosis, myocardial infarction, ischemia reperfusion, Reynauld's syndrome, inflammatory bowel disease, osteoarthritis, acute respiratory distress syndrome, asthma, chronic obstructive pulmonary disease (COPD), emphysema, lung inflammation, delayed type hyper-sensitivity reaction, idiopathic pulmonary fibrosis, cystic fibrosis, thermal injury, stroke, experimental allergic encephalomyelitis, multiple organ injury syndrome secondary to trauma, neutrophilic dermatosis (Sweet's disease), glomerulonephritis, ulcerative colitis, Crohn's disease, necrotizing enterocolitis, cytokine-induced toxicity, gingivitis, periodontitis, hemolytic uremic syndrome, psoriasis, systemic lupus erythematosus, autoimmune thyroiditis, multiple sclerosis, rheumatoid arthritis, Grave's disease, immunological-mediated side effects of treatment associated with hemodialysis or leukapheresis, granulocyte transfusion associated syndrome, deep vein thrombosis, post-thrombotic syndrome, unstable angina, transient ischemic attacks, peripheral vascular disease, (e.g., peripheral artery disease), metastasis associated with cancer, sickle syndromes, including but not limited to sickle cell anemia, organ rejection (graft vs. host), or congestive heart failure.

In some embodiments, the disease, disorder, condition, or undesired process can be an undesired infection process mediated by a bacteria, a virus, or a parasite, for example gingivitis, periodontitis, hemolytic uremic syndrome, or granulocyte transfusion associated syndrome.

In some embodiments, the disease, disorder, condition, or undesired process can be metastasis associated with cancer. In further embodiments, the disease, disorder, condition, or undesired process can be a disease or disorder associated with an undesired immunological process, for example psoriasis, systemic lupus erythematosus, autoimmune thyroiditis, multiple sclerosis, rheumatoid arthritis, Grave's disease, and immunological-mediated side effects of treatment associated with hemodialysis or leukapheresis. In certain embodiments, the disease, disorder, condition, or undesired process can be a condition associated with an undesired thrombotic process, for example, deep vein thrombosis, unstable angina, transient ischemic attacks, peripheral vascular disease, post-thrombotic syndrome, venous thromboembolism, or congestive heart failure.

In some embodiments, the present teachings provide methods of ameliorating an undesired immunological process in a transplanted organ (e.g., renal transplant) that include administering to the organ a compound of the present teachings or its pharmaceutically acceptable salt, hydrate, or ester. In some embodiments, the present teachings provide methods of treating, or ameliorating a symptom of a sickle syndrome, for example, sickle cell anemia, that include administering a compound of the present teachings to a patient in need thereof. In some embodiments, the methods can include identifying a human, mammal or animal that has a biomarker for a disease or disorder involving selectin-mediated intracellular adhesion, and administering to the human, mammal or animal a therapeutically effective amount of a compound described herein. In some embodiments, the biomarker can be one or more of soluble P-selectin, CD40, CD 40 ligand, MAC-1, TGF beta, ICAM, VCAM, IL-1. IL-6, IL-8, Eotaxin, RANTES, MCP-1, PIGF, CRP, SAA, and platelet monocyte aggregates.

The compounds of the present teachings may be prepared by means of known methods. In particular, compounds of the present teachings can be prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (NMR, e.g., ₁H or ₁₃C), infrared spectroscopy (IR), spectrophotometry (e.g., UV-visible), mass spectrometry (MS), or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents, which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

Compounds of the present teachings can by synthesized generally according to Schemes 1-6.

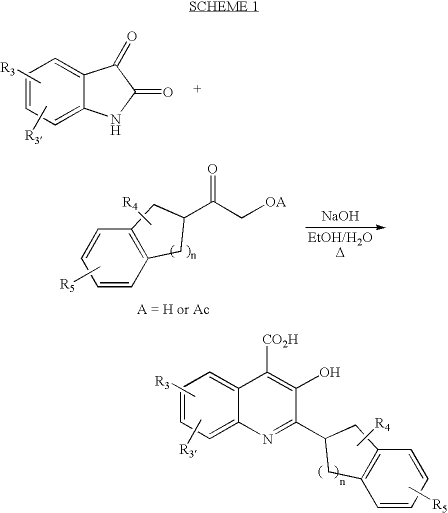

Compounds of the present teachings can be prepared by reacting an optionally substituted indoline-2,3-dione with an optionally substituted 2-oxo-ethyl acetate or corresponding alcohol in the presence of a base, e.g. NaOH, as shown above in Scheme 1, wherein R₃, R_{3′}, R₄, R₅, and n are as defined herein.

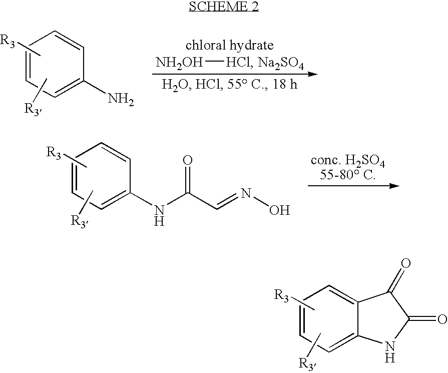

The substituted indoline-2,3-dione can be prepared from an appropriately substituted aniline as shown above in Scheme 2, wherein R₃ and R_{3′} are as defined herein.

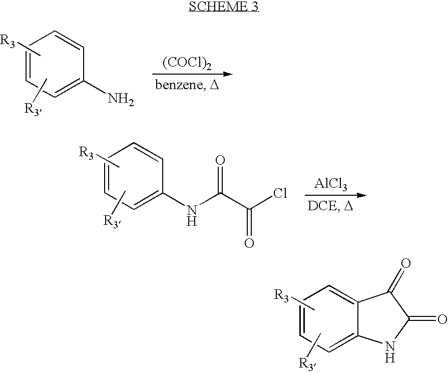

Alternatively, the substituted indoline-2,3-dione can be prepared from an appropriately substituted aniline as shown above in Scheme 3, wherein R₃ and R_{3′} are as defined herein.

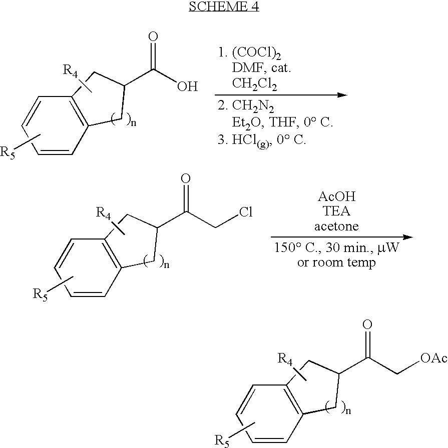

The substituted 2-oxo-ethyl acetate can be prepared from an appropriately substituted carboxylic acid as shown above in Scheme 4, wherein R₄, R₅, and n are as defined herein.

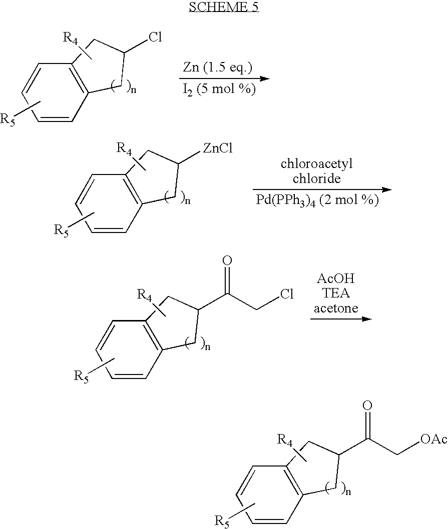

Alternatively, the substituted 2-oxo-ethyl acetate can be prepared from an appropriately substituted halide, as shown above in Scheme 5, wherein R₄, R₅, and n are as defined herein.

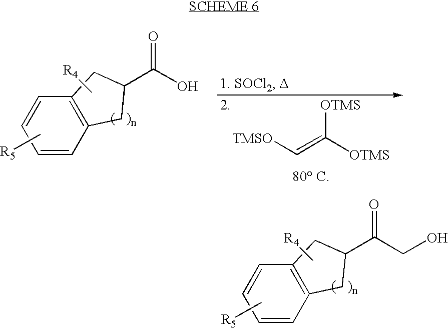

Alternatively, the corresponding alcohol of the substituted 2-oxo-ethyl acetate can be prepared from the appropriately substituted carboxylic acid as shown above in Scheme 6, wherein R₄, R₅, and n are as defined herein.

### Preparation of Exemplified Compounds

The following non-limiting examples are presented merely to illustrate the present teachings. A skilled person in the art will understand that there are numerous equivalents and variations that are not exemplified but still form part of the present teachings.

### EXAMPLE 1

### PREPARATION OF 2-(1,2-DIHYDROCYCLOBUTABENZEN-1-YL)-3-HYDROXY-8-(TRIFLUOROMETHYL)QUINOLINE-4-CARBOXYLIC ACID (COMPOUND 1)

### Step 1: Preparation of 1-(1,2-dihydrocyclobutabenzen-1-yl)-2-hydroxyethanone

A mixture of 1-benzocyclobutenecarboxylic acid (1.0 grams (g), 6.76 millimolar (mmol)) and 3.5 mL of thionyl chloride in 15 milliliter (mL) of toluene was heated at 115° C. for 16 hours (hrs). Concentration of the reaction mixture gave an oily residue. To this residue was added 10 mL of toluene and the resulting mixture was concentrated to yield a yellow oil, to which was added 1,1,2-tris(trimethylsilyloxy)ethane (4.4 mL, 13.34 mmol). The resulting mixture was heated at 100° C. for 16 hours under nitrogen atmosphere. The reaction mixture was cooled to 50° C. and to it were added 10 mL of dioxane and 2 mL of 1 Normal (N) HCl. The resulting mixture was stirred at 80° C. for 2 hours. Concentration of the mixture gave a yellow oily residue, to which 10 mL of water and 15 mL of diethyl ether were added. The organic layer was washed with 5 mL of saturated sodium bicarbonate solution, brine, and dried over magnesium sulfate. The solid was removed via filtration. Concentration of the filtrate afforded 1-(1,2-dihydrocyclobutabenzen-1-yl)-2-hydroxyethanone (0.55 g, 65% yield) as a colorless oil. ₁H NMR (400 MHz, CDCl₃) δ2.82-2.98 (m, 1 H), 3.05-3.20 (m, 1 H), 3.46-3.51 (m, 1 H), 4.44-4.47 (m, 2 H), 7.05-7.81 (m, 4 H).

### Step 2: Preparation of 2-(1,2-dihydrocyclobutabenzen-1-yl)-3-hydroxy-8-(trifluoromethyl)quinoline-4-carboxylic acid (Compound 1)

General procedures for the Pfitzinger reaction described by Cragoe et al. (see, J. Org. Chem., 1953, 18: 561) was followed. To a mixture of 7-(trifluoromethyl)indoline-2,3-dione (130.0 milligram (mg), 0.60 mmol) in 0.5 mL of ethanol and 1 mL of aqueous 6 M potassium hydroxide solution at 100° C. was added a warm solution of 1-(1,2-dihydrocyclobutabenzen-1-yl)-2-hydroxyethanone (Example 1, 100 mg, 0.62 mmol) in 0.5 mL of ethanol in small portions over 0.5-hour period. After the addition was completed, the reaction mixture was heated at reflux temperature until HPLC-MS indicated the reaction was complete (varying from 1 hour to 16 hours). Solvent was removed and the crude product was purified by preparative HPLC. Fractions containing2-(1,2-dihydrocyclobutabenzen-1-yl)-3-hydroxy-8-(trifluoromethyl)quinoline-4-carboxylic acid salt were concentrated. The resulting solid was dissolved in 1 mL of acetonitrile and the resulting solution was acidified with concentrated hydrochloric acid to pH ˜1 at 0° C. Water (20 mL) was added and the resulting suspension was stirred vigorously at 0° C. for 1 hour. The yellow solid was collected via filtration, washed with water, and dried under vacuum to yield 2-(1,2-dihydrocyclobutabenzen-1-yl)-3-hydroxy-8-(trifluoromethyl)quinoline-4-carboxylic acid (12.5 mg, 5.8% yield) as a light yellow solid. ₁H NMR (400 MHz, methanol-d₄ “MeOH-d₄”) δ3.46 (dd, J=14.0, 5.6 Hz, 1 H), 3.98 (dd, J=14.0, 3.1 Hz, 1 H), 5.06 (dd, J=5.6, 3.1 Hz, 1 H), 6.99 (d, J=6.3 Hz, 1 H), 7.04-7.09 (m, 2 H), 7.25 (d, J=6.1 Hz, 1 H), 7.37-7.39 (m, 1H), 7.62 (d, J=7.3 Hz, 1 H), 9.50 (d, J=8.6 Hz, 1 H).

### EXAMPLE 2

### PREPARATION OF 2-(1,2-DIHYDROCYCLOBUTABENZEN-1-YL)-3-HYDROXY-7,8-DIMETHYLQUINOLINE-4-CARBOXYLIC ACID (COMPOUND 2)

### Step 1: Preparation of 6,7-dimethyl-1H-indole-2,3-dione

The isatin synthesis described by Rewcastle et al. (see, J. Med. Chem., 1991, 34: 217) was used. Chloral hydrate (45 g, 0.27 mol), hydroxylamine hydrochloride (205 g, 1.25 mol), and sodium sulfate (226.5 g, 1.6 mol) were placed in a 2 L round-bottom flask and 750 mL of water was added. To this suspension was added 2,3-dimethyl aniline (29.05 g, 0.24 mol) in 250 mL of water containing 25 mL of concentrated HCl. The suspension was heated at 45° C. under nitrogen atmosphere for 90 minutes (min.), then at 52° C. for 45 minutes, and finally at 75° C. for 60 minutes. The reaction mixture was cooled to room temperature. The precipitate was collected by filtration, washed with water and petroleum ether, and dried overnight in a vacuum desiccators to give N-(2,3-dimethyl-phenyl)-2-hydroxyimino-acetamide (40.1 g, 87% yield).

N-(2,3-Dimethyl-phenyl)-2-hydroxyimino-acetamide (20 g, 0.1 mol) was added in small portions, with stirring, to 80 mL of CH₃SO₃H at 70° C.-80° C. in one hour. The resulting mixture was left at the same temperature for 15 minutes and was poured onto crushed ice in a beaker. Additional ice was added until the outside of the beaker felt cold to touch. The precipitate was collected and dissolved in 1 N aqueous sodium hydroxide solution. Neutralization with acetic acid precipitated impurities which were removed by filtration and acidification with hydrochloric acid of the filtrate gave 6,7-dimethyl-1H-indole-2,3-dione as a solid (12.8 g, 70% yield). ₁H NMR (400 MHz, dimethylsulfoxide-d₆ “DMSO-d₆”) δ2.09 (s, 3 H), 2.27 (s, 3 H), 6.89 (d, J=7.58 Hz, 1 H), 7.25 (d, J=7.58 Hz, 1 H), 11.02 (s, 1 H).

### Step 2: Preparation of 2-(1,2-dihydrocyclobutabenzen-1-yl)-3-hydroxy-7,8-dimethylquinoline-4carboxylic acid (Compound 2)

2-(1,2-Dihydrocyclobutabenzen-1-yl)-3-hydroxy-7,8-dimethylquinoline-4-carboxylic acid was synthesized following the procedures described in Example 1 by reacting 6,7-dimethylindoline-1H-2,3-dione (Example 2, 105.0 mg, 0.60 mmol) with 1-(1,2-dihydrocyclobutabenzen-1-yl)-2-hydroxyethanone (Example 1, 100 mg, 0.62 mmol), and was obtained as a yellow solid (1.5 mg, 0.78% yield). ₁H NMR (400 MHz, MeOH-d₄) δ2.62-2.66 (s, 3 H), 2.85-2.90 (s, 3 H), 3.87 (dd, J=14.0, 5.6 Hz, 1 H), 4.11 (dd, J=14.0, 3.1 Hz, 1 H), 5.41 (dd, J=5.6, 3.1 Hz, 1 H), 7.32-7.37 (m, 1 H), 7.40-7.45 (m, 2 H), 7.56 (d, J=8.7 Hz, 2 H), 8.75 (d, J=8.7 Hz, 1 H).

### EXAMPLE 3

### PREPARATION OF 3-HYDROXY-2-INDAN-2-YL-7,8-DIMETHYL-QUINOLINE-4-CARBOXYLIC ACID (COMPOUND 3)

### Step 1. Preparation of 2-hydroxy-1-indan-2-yl-ethanone

A mixture of indan-2-carboxylic acid (1.0 g, 6.2 mmol) and 3.5 milliliter (mL) of thionylchloride in 7.5 mL of toluene was heated at 115° C. for 16 hours. Concentration of the reaction mixture gave an oily residue. To this residue was added 10 mL of toluene and the resulting mixture was concentrated to yield a yellow oil to which 1,1,2-tris(trimethylsilyloxy)ethane (4.1 mL, 12.4 mmol) was added. The reaction mixture was heated at 100° C. for 16 hours under nitrogen atmosphere. The reaction mixture was cooled to 50° C. and to it were added 5 mL of dioxane and 1 mL of aqueous HCl solution. The resulting mixture was stirred at 80 ° C. for 2 hours and concentration of the mixture gave a yellow oily residue. The residue was partitioned between 10 mL of water and 15 mL of diethyl ether. The organic layer was washed with 5 mL of saturated sodium bicarbonate solution, brine, and dried over magnesium sulfate. Solids were removed via filtration and the filtrate was concentrated to afford 2-hydroxy-1-indan-2-yl-ethanone (0.80 g, 73% yield)0 as a colorless oil. ₁H NMR (400 MHz, CDCI₃) 63.12-3.24 (m, 4 H), 3.41-3.51 (m, 1 H), 4.84-4.86 (d, J=4.55 Hz, 2 H), 7.16-7.25 (m, 4 H).

### Step 2: Preparation of 3-hydroxy-2-indan-2-yl-7,8-dimethyl-quinoline-4-carboxylic acid (Compound 3)

Following the procedures described in Example 1,6,7-dimethylindoline-2,3-dione (Example 2, 90 mg, 0.51 mmol) was reacted with 2-hydroxy-1-indan-2-yl-ethanone (Example 3, 90 mg, 0.51 mmol) in the presence of 6 M KOH. 3-Hydroxy-2-indan-2-yl-7,8-dimethyl-quinoline-4-carboxylic acid was obtained as a beige solid 18.2 mg, 10.7% yield). ₁H NMR (400 MHz, DMSO-d₆) δ2.38 (s, 3 H), 2.59 (m, 3 H), 3.35 (dd, J=15.41, 8.59 Hz, 2 H), 3.44 (dd, J=15.41, 7.58 Hz, 2 H), 4.25-4.35 (m, 1 H), 7.13-7.17 (m, 2 H), 7.24-7.29 (m, 2 H), 7.36 (d, J=8.84 Hz, 1 H), 8.29 (d, J=8.84 Hz, 1 H).

### EXAMPLE 4

### PREPARATION OF 3-HYDROXY-2-INDAN-2-YL-8-ISOPROPYL-QUINOLINE-4-CARBOXYLIC ACID (COMPOUND 4)

### Step 1: Preparation of 7-isopropyl indole-2,3-dione

7-Isopropyl indole-2,3-dione was prepared following the procedures described in Example 3 for the preparation of 2-hydroxy-1-indan-2-yl-ethanone and was obtained as a brown powder (46% yield). ₁H NMR (400 MHz, DMSO-d₆) δ1.18 (d, J=6.8 Hz, 6 H), 3.04 (sep, 1 H), 7.06 (t, J=7.7 Hz, 1 H), 7.35 (d, J=7.3 Hz, 1 H), 7.54 (d, J=7.3 Hz, 1 H), 11.09 (s, 1 H).

### Step 2: Preparation of 3-hydroxy-2-indan-2-yl-8-isopropyl-quinoline-4-carboxylic acid (Compound 4)

Following the procedures described in Example 1, 7-isopropylindoline-2,3-dione (Example 4, 189 mg, 1.0 mmol) was reacted with 2-hydroxy-1-indan-2-yl-ethanone (Example 3, 171 mg, 1.0 mmol) to provide 3-hydroxy-2-indan-2-yl-8-isopropyl-quinoline-4-carboxylic acid (40.4 mg, 11.6% yield) as a beige solid. ₁H NMR (400 MHz, DMSO-d₆) δ1.22 (d, J=6.82 Hz, 6 H), 3.34-3.41 (m, 4 H), 4.03-4.14 (m, 1 H), 4.27-4.37 (m, 1 H), 7.12-7.16 (m, 2 H), 7.24-7.28 (m, 2 H), 7.38 (d, J=7.37 Hz, 1 H), 7.48 (dd, J=8.34, 7.37 Hz, 1 H), 8.36 (d, J=8.34 Hz, 1 H).

### EXAMPLE 5

### PREPARATION OF 3-HYDROXY-2-INDAN-2-YL-8-TRIFLUOROMETHYL-QUINOLINE-4-CARBOXYLIC ACID (COMPOUND 5)

### Step 1: Preparation of 7-trifluoromethyl-1H-indole-2,3-dione

7-Trifluoromethyl-1H-indole-2,3-dione was prepared following the procedures in Example 2 for the preparation of 6,7-dimethyl-1H-indole-2,3-dione and was obtained as a solid (61% yield). ₁H NMR (400 MHz, DMSO-d₆) δ7.23 (t, J=7.7 Hz, 1 H), 7.78 (d, J=7.3 Hz, 1 H), 7.85 (d, J=8.1 Hz, 1 H), 11.46 (s, 1 H).

### Step 2: Preparation of 3-hydroxy-2-indan-2-yl-8-trifluoromethyl-quinoline-4-carboxylic acid (Compound 5)

Following the procedures described in Example 1, 7-(trifluoromethyl)indoline-2,3-dione (Example 5, 313 mg, 1.46 mmol) was reacted with 2-hydroxy-1-indan-2-yl-ethanone (Example 3, 257 mg, 1.46 mmol) to yield 3-hydroxy-2-indan-2-yl-8-trifluoromethyl-quinoline-4-carboxylic acid (87.8 mg, 16.1% yield) as a beige solid. ₁H NMR (400 MHz, DMSO-d₆) δ3.34 (dd, J=15.66, 8.59 Hz, 2 H), 3.43 (dd, J=15.66, 8.08 Hz, 2 H), 4.26-4.38 (m, 1 H), 7.10-7.16 (m, 2 H), 7.22-7.28 (m, 2 H), 7.65 (dd, J=8.94, 7.88 Hz, 1 H), 7.88 (d, J=7.88 Hz, 1 H), 8.95 (d, J=8.94 Hz, 1 H).

### Biological Test

### BIACORE P-SELECTIN/PSGL-1 INHIBITION ASSAY

Surface plasmon resonance assays were performed on a Biacore 3000 instrument (Biacore Inc. Piscataway, N.J.) at 25° C. at a flow rate of 30 μL/minute and each assay consisted of a 60-second equilibration, a 60-μL sample injection (kinject), and a 300-second dissociation.

A purified, monomeric, truncated form of human PSGL-1, “19ek”, that contained all the necessary P-selectin binding determinants (see Goetz, et al., J Cell Biol., 1997, 137: 509-519; and Sako, et al., Cell, 1995, 83: 323-331) was biotinylated via amine chemistry (Sulfo-NHS-LC-Biotin, Peirce) at a unique C-terminal lysine residue (see Somers, et al., Cell, 2000, 103: 467-479) and immobilized on a Biacore SA sensor chip (Biacore Inc.), using an HBS-EP buffer (Biacore Inc.), and the target 600-700 RU. The coated chip was re-equilibrated with an HBS-P buffer (Biacore Inc.) to which 1 mM CaCl₂ and 1 mM MgCl₂ (both from Fisher) were added to ensure sufficient calcium for the calcium-dependent interaction between the receptor and the ligand.

Test compounds were incubated for 1 hour in a 1.1× Biacore assay buffer. Each solution was centrifuged through a 0.2 μm filter, using a 96-well plate format (Millipore). Glycyrrhizin tri-sodium salt (TCl) was prepared as a positive control in parallel with the test compounds, in the same manner described above. Glycyrrhizin, a demonstrated antagonist of P-selectin (see Patton, J. T., GlycoTech Corporation, written communication, May 2000), has been shown to inhibit the P-selectin/PSGL-1 interaction with an IC₅₀ of 1 mM in this assay.

A soluble recombinant truncated form of human P-selectin, P-LE, comprised of the lectin and epidermal growth factor-like (EGF) domains expressed in CHO cells (see Somers, et al., Cell, 2000, 103: 467-479) was added to each filtered test compound solution. Final concentrations of reagents were 500 nM P.LE, 250 or 500 μM test compound (depending on structure) or 1 mM glycyrrhizin, 10% DMSO, and 1× Biacore buffer (100 mM HEPES, 150 mM NaCl, 1 mM CaCl₂, and 1 mM MgCl₂ (all reagents from Fisher)), with a pH of 7.4. Compounds active at 250 μM were titrated to further define activity. Test samples were supplied to the Biacore instrument in a 96-well plate.

The Biacore raw data file was exported as a text file to an Excel spreadsheet, where the buffer blanks bracketing the samples were averaged for each Biacore instrument flow cell (Fc), and subtracted from the averaged uninhibited P.LE samples and from all the other samples. The reference signal from Fc1 (uncoated) was then subtracted from its corresponding active (coated) signal for each injection, a process known as double referencing (see Myszka, J Mol. Recognit., 1999, 12(5): 279-284). The percent inhibition of binding was calculated by dividing the reference-subtracted inhibited signal by the reference-subtracted uninhibited signal, subtracting this value from 1, and multiplying the resulting value by 100. The replicate percent inhibition values were averaged and expressed as the mean±standard deviation. The inter-experiment standard deviation of calculated percent inhibitions in the Biacore assay was ±5.

Assay results for representative compounds according to the invention are included in Table 1 below.

TABLE 1

| | | | % inhibition |
|---|---|---|---|
| Compound | Structure | Name | at 250 uM |
| 1 | 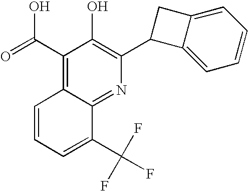 | 2-(1,2-dihydrocyclobutabenzen-1-yl)-3-hydroxy-8-(trifluoro-methyl)quinoline-4-carboxylic acid | 45 |
| 2 | 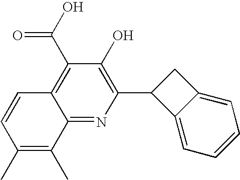 | 2-(1,2-dihydrocyclobutabenzen-1-yl)-3-hydroxy-7,8-dimethyl-quinolin-4-carboxylic acid | 17 |
| 3 | 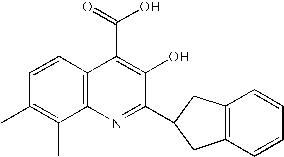 | 3-hydroxy-2-indan-2-yl-7,8-dimethyl-quinolin-4-carboxylicacid | 11 |
| 4 | 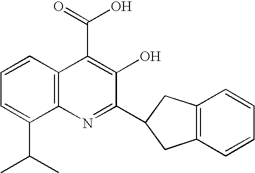 | 3-hydroxy-2-indan-2-yl-8-isopropyl-quinolin-4-carboxylicacid | 48 |
| 5 | 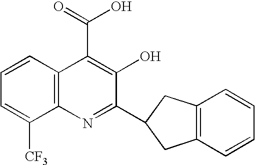 | 3-hydroxy-2-indan-2-yl-8-trifluoromethyl-quinoline-4-carboxylic acid | 35 |

As those skilled in the art will appreciate, numerous changes and modifications can be made to the preferred embodiments of the present teachings without departing from the spirit of the present teachings. It is intended that all such variations fall within the scope of the present teachings.

What is claimed is:

1. A compound of formula I: 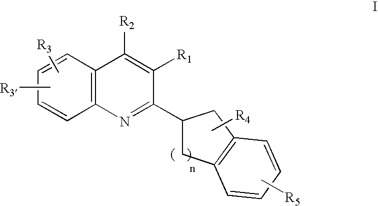
or a pharmaceutically acceptable salt, hydrate, or ester thereof, wherein:
R₁ is —OR₆, —C(O)R₇, —C(O)OR₆, —C(O)NR₇R₈, —C(S)R₇, —C(S)OR₆, —C(S)NR₇R₈, —C(NR₇)R₇, —C(NR₇)NR₇R₈, —NR₇R₈, —NR₈C(O)R₇, —NR₈C(O)NR₇R₈, —NR₈C(NR₇)NR₇R₈, —NR₈S(O)ₘR₇, or —NR₈S(O)ₘNR₇R₈;
R₂ is —C(O)OR₆, —C(O)NR₇R₈, or a carboxylic acid bioisostere;
R₃ and R₃ independently are H, —CN, —NO₂, halogen, —OR₆, —NR₇R₈, —S(O)ₘR₇, —S(O)ₘOR₆, —S(O)ₘNR₇R₈, —C(O)R₇, —C(O)OR₆, —C(O)NR₇R₈, —C(S)R₇, —C(S)OR₆, —C(S)NR₇R₈, —C(NR₇)NR₇R₈, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₉ groups; or
alternatively, R₃ and R_{3′}, together with the carbon atoms to which each is attached, form a C₄₋₁₄ cycloalkyl group, a C₆₋₁₄ aryl group, a 4-14 membered cycloheteroalkyl group, or a 5-14 membered heteroaryl group, wherein each of the C₄₋₁₄ cycloalkyl group, the C₆₋₁₄ aryl group, the 4-14 membered cycloheteroalkyl group, and the 5-14 membered heteroaryl group optionally is substituted with 1-4-Z-R₉ groups;
R₄ and R₅ independently are H, —CN, —NO₂, halogen, —OR₆, —NR₇R₈, —S(O)ₘR₇, —S(O)ₘOR₆, —S(O)ₘNR₇R₈, —C(O)R₇, —C(O)OR₆, —C(O)NR₇R₈, —C(S)R₇, —C(S)OR₆, —C(S)NR₇R₈, —C(NR₇)NR₇R₈, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, C₂₋₁₀ alkenyl group, C₂₋₁₀ alkynyl group, C₃₋₁₄ cycloalkyl group, C₆₋₁₄ aryl group, 3-14 membered cycloheteroalkyl group, and 5-14 membered heteroaryl group, optionally is substituted with 1-4 -Z-R₉ groups;
R₆, at each occurrence, independently is H, —C(O)R₇, —C(O)NR₇R₈, —C(S)R₇, —C(S)NR₇R₈, —C(NR₇)R₇, —C(NR₇)NR₇R₈, —S(O)ₘR₇, —S(O)ₘNR₇R₈, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₉ groups;
R₇ and R₈, at each occurrence, independently are H, —OH, —SH, —S(O)₂OH, —C(O)OH, —C(O)NH₂, —C(S)NH₂, —OC₁₋₁₀ alkyl, —C(O)-C₁₋₁₀ alkyl, —C(O)—OC₁₋₁₀ alkyl, —OC₆₋₁₄ aryl, —C(O)—C₆₋₁₄ aryl, —C(O)—OC₆₋₁₄ aryl, —C(S)N(C₁₋₁₀ alkyl)₂, —C(S)NH—C₁₋₁₀ alkyl, —C(O)NH—C₁₋₁₀ alkyl, —C(O)N(C₁₋₁₀ alkyl)₂, —C(O)NH—C₆₋₁₄ aryl, —S(O)ₘ—C₁₋₁₀ alkyl, —S(O)ₘ—OC₁₋₁₀ alkyl, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₉ groups;
R₉, at each occurrence, independently is halogen, —CN, —NO₂, oxo, —O-Z-R₁₀, —NR₁₀-Z-R₁₁, —N(O)R₁₀-Z-R₁₁, —S(O)ₘR₁₀, —S(O)ₘO-Z-R₁₀, S(O)ₘNR₁₀-Z-R₁₁, —C(O)R₁₀, —C(O)O-Z-R₁₀, —C(O)NR₁₀-Z-R₁₁, —C(S)NR₁₀-Z-R₁₁, —Si(C₁₋₁₀ alkyl)₃, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4 R₁₂ groups;
R₁₀ and R₁₁, at each occurrence, independently are H, —OH, —SH, —S(O)₂OH, —C(O)OH, —C(O)NH₂, —C(S)NH₂, —OC₁₋₁₀ alkyl, —C(O)—C₁₋₁₀ alkyl, —C(O)—OC₁₋₁₀ alkyl, —C(S)N(C₁₋₁₀ alkyl)₂, —C(S)NH—C₁₋₁₀ alkyl, —C(O)NH—C₁₋₁₀ alkyl, —C(O)N(C₁₋₁₀ alkyl)₂, —S(O)ₘ—C₁₋₁₀ alkyl, —S(O)ₘ—OC₁₋₁₀ alkyl, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R₁₂ groups;
R₁₂, at each occurrence, independently is halogen, —CN, —NO₂, oxo, —OH, —NH₂, —NH(C₁₋₁₀ alkyl), —N(C₁₋₁₀ alkyl)₂, —S(O)ₘH, —S(O)ₘ—C₁₋₁₀ alkyl, —S(O)₂OH, —S(O)ₘ—OC₁₋₁₀ alkyl, —CHO, —C(O)—C₁₋₁₀ alkyl, —C(O)OH, —C(O)—OC₁₋₁₀ alkyl, —C(O)NH₂, —C(O)NH—C₁₋₁₀ alkyl, —C(O)N(C₁₋₁₀ alkyl)₂, —C(S)NH₂, —C(S)NH—C₁₋₁₀ alkyl, —C(S)N(C₁₋₁₀ alkyl)₂, —S(O)ₘNH₂, —S(O)ₘNH(C₁₋₁₀ alkyl), —S(O)ₘN(C₁₋₁₀ alkyl)₂, —Si(C₁₋₁₀ alkyl)₃, a C₁₋₁₀ alkyl group, a C₂₋₁₀ alkenyl group, a C₂₋₁₀ alkynyl group, a C₁₋₁₀ alkoxy group, a C₁₋₁₀ alkylthio group, a C₁₋₁₀ haloalkyl group, a C₃₋₁₄ cycloalkyl group, a C₆₋₁₄ aryl group, a 3-14 membered cycloheteroalkyl group, or a 5-14 membered heteroaryl group;
Z, at each occurrence, independently is a divalent C₁₋₁₀ alkyl group, a divalent C₂₋₁₀ alkenyl group, a divalent C₂₋₁₀ alkynyl group, a divalent C₁₋₁₀ haloalkyl group, or a covalent bond;
m, at each occurrence, independently is 0, 1, or 2; and
n is 0, 1, or 2.

2. The compound of claim 1 or a pharmaceutically acceptable salt, hydrate, or ester thereof, wherein R₁ is —OR₆, —OC(O)R₇, or —NR₇R₈; wherein R₆, R₇, and R₈ are defined in claim 1.

3. The compound of claim 2 or a pharmaceutically acceptable salt, hydrate, or ester thereof, wherein R₁ is —OH.

4. The compound of claim 1 or a pharmaceutically acceptable salt, hydrate, or ester thereof, wherein R₂ is —COOH.

5. The compound of claim 1 or a pharmaceutically acceptable salt, hydrate, or ester thereof, wherein the compound has formula Ia, formula Ib, formula Ic, formula Id, formula Ie, or formula If: 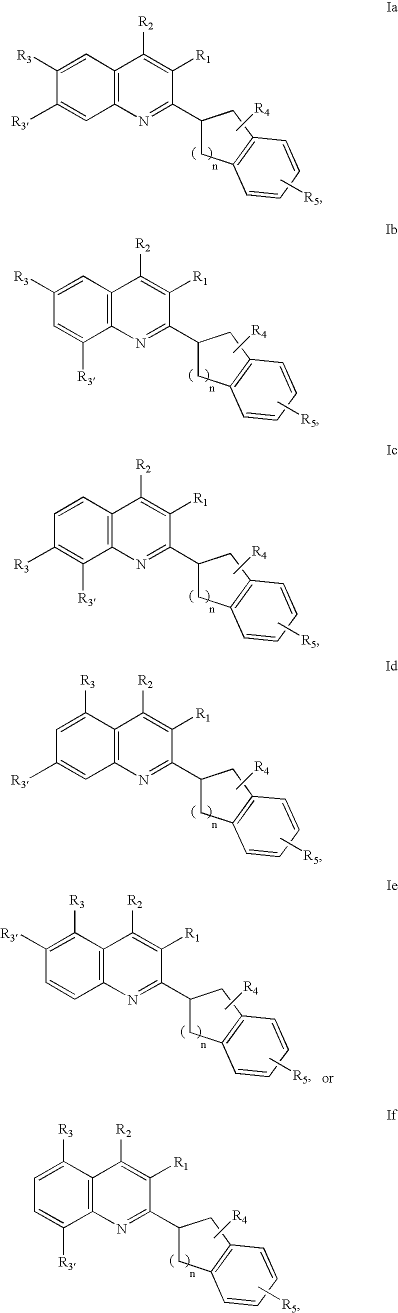
wherein R₁, R₂, R₃, R_{3′}, R₄, R₅ and n are as defined in claim 1.

6. The compound of claim 1 or a pharmaceutically acceptable salt, hydrate, or ester thereof, wherein R₃ and R_{3′} are each independently H, halogen, —OR₆, a C₁₋₁₀ alkyl group, or a C₆₋₁₄ aryl group, wherein each of the C₁₋₁₀ alkyl group and the C₆₋₁₄ aryl group optionally is substituted with 1-4-Z-R₉ groups and Z and R₉ are as defined in claim 1.

7. The compound of claim 1 or a pharmaceutically acceptable salt, hydrate, or ester thereof, wherein R₃ and R_{3′} independently are H, halogen, —CF₃, a C₁₋₁₀ alkyl group, a C₃₋₁₄cycloalkyl group, —CO₂H, —OC₁₋₁₀alkyl, —OCF₃, —C(CF₃)₂OH, phenyl, or 5-14 membered heteroaryl group.

8. The compound of claim 1 or a pharmaceutically acceptable salt, hydrate, or ester thereof, wherein one of R₃ and R_{3′} is H and the other is —CF₃.

9. The compound of claim 1 or a pharmaceutically acceptable salt, hydrate, or ester thereof, wherein R₃ and R_{3′}, together with the carbon atoms to which each is attached, form a C₄₋₁₄ cycloalkyl group or a 4-14 membered cycloheteroalkyl group, wherein each of the C₄₋₁₄ cycloalkyl group and the 4-14 membered cycloheteroalkyl group optionally is substituted with 1-4-Z-R₉ groups and Z and R₉ are as defined in claim 1.

10. The compound of claim 9, or a pharmaceutically acceptable salt, hydrate, or ester form thereof, wherein the compound has formula Ig: 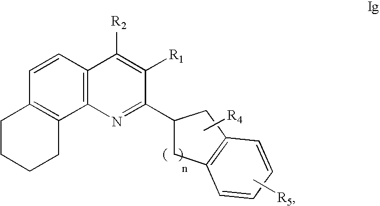
wherein R₁, R₂, R₄, R₅, and n are as defined in claim 1.

11. The compound of claim 1 or a pharmaceutically acceptable salt, hydrate, or ester thereof, wherein R₄ is H, —CN, —NO₂, halogen, —OH, —NH₂, —C(O)OH, —C(O)NH₂, —O(C₁₋₁₀ alkyl), —NH(C₁₋₁₀ alkyl), —N(C₁₋₁₀ alkyl)₂, —C(O)O(C₁₋₁₀ alkyl), —C(O)NH(C₁₋₁₀ alkyl), —C(O)N(C₁₋₁₀ alkyl)₂, or a C₁₋₁₀ alkyl group optionally substituted with 1-4-Z-R₉ groups; wherein R₉ and Z are as defined in claim 1.

12. The compound of claim 1 or a pharmaceutically acceptable salt, hydrate, or ester thereof, wherein R₅ is H, —CN, —NO₂, halogen, —OH, —NH₂, —C(O)OH, —C(O)NH₂, —O(C₁₋₁₀ alkyl), —NH(C₁₋₁₀ alkyl), —N(C₁₋₁₀ alkyl)₂, —C(O)O(C₁₋₁₀ alkyl), —C(O)NH(C₁₋₁₀ alkyl), —C(O)N(C₁₋₁₀ alkyl)₂, or a C₁₋₁₀ alkyl group optionally substituted with 1-4-Z-R₉ groups; wherein R₉ and Z are as defined in claim 1.

13. The compound of claim 1 or a pharmaceutically acceptable salt, hydrate, or ester thereof, wherein the compound has formula IIa or IIb: 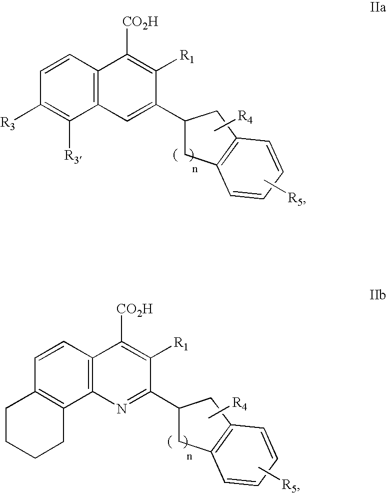
wherein R₁, R₃, R_{3′}, R₄, R₅, and n are as defined in claim 1.

14. The compound of claim 1 or a pharmaceutically acceptable salt, hydrate, or ester thereof, wherein n is 0.

15. The compound of 1 or a pharmaceutically acceptable salt, hydrate, or ester thereof, wherein n is 1.

16. A compound of claim 1 wherein the compound is selected from 2-(1,2-dihydrocyclobutabenzen-1-yl)-3-hydroxy-8-(trifluoromethyl)quinoline-4-carboxylic acid; 2-(1,2-dihydrocyclobutabenzen-1-yl)-3-hydroxy-7,8-dimethylquinoline-4-carboxylic acid; 3-hydroxy-2-indan-2-yl-7,8-dimethyl-quinoline-4-carboxylic acid; 3-hydroxy-2-indan-2-yl-8-isopropyl-quinoline-4-carboxylic acid; and 3-hydroxy-2-indan-2-yl-8-trifluoromethyl-quinoline-4-carboxylic acid; or a pharmaceutically acceptable salt, hydrate, or ester thereof.

17. A pharmaceutical composition comprising a therapeutically effective amount of a compound of claim 1 or a pharmaceutically acceptable salt, hydrate, or ester thereof, and a pharmaceutically acceptable carrier or excipient.

18. A method of inhibiting selectin-mediated intracellular adhesion in a mammal comprising administering to said mammal a therapeutically effective amount of a compound of claim 1 or a pharmaceutically acceptable salt, hydrate, or ester thereof.

19. A method of treating a disease, complications of a disease, a disorder, condition, or undesired process in a mammal, said method comprising administering to said mammal a compound of claim 1, wherein said disease, disorder, condition, or undesired process is selected from atherosclerosis, restenosis, myocardial infarction, ischemia reperfusion, Reynauld's syndrome, inflammatory bowel disease, osteoarthritis, acute respiratory distress syndrome, asthma, chronic obstructive pulmonary disease (COPD), emphysema, lung inflammation, delayed type hypersensitivity reaction, idiopathic pulmonary fibrosis, cystic fibrosis, thermal injury, stroke, experimental allergic encephalomyelitis, multiple organ injury syndrome secondary to trauma, neutrophilic dermatosis (Sweet's disease), glomerulonephritis, ulcerative colitis, Crohn's disease, necrotizing enterocolitis, cytokine-induced toxicity, gingivitis, periodontitis, hemolytic uremic syndrome, psoriasis, systemic lupus erythematosus, autoimmune thyroiditis, multiple sclerosis, rheumatoid arthritis, scleritis, Grave's disease, immunological-mediated side effects of treatment associated with hemodialysis or leukapheresis, granulocyte transfusion associated syndrome, deep vein thrombosis, post-thrombotic syndrome, unstable angina, transient ischemic attacks, peripheral vascular disease, metastasis associated with cancer, sickle cell anemia, organ transplant rejection and congestive heart failure.
